(19) United States
(12) Patent Application Publication
(10) Pub. No.: US 2010/0152665 A1
(43) Pub. Date: Jun. 17, 2010

(54) Cannula Insertion Device with Automatic Needle Retraction Comprising Only One Spring

(76) Inventor: Søren Bo HASTED, Copenhagen O (DK)

Correspondence Address: BRINKS HOFER GILSON & LIONE, P.O. BOX 10395, CHICAGO, IL, 60610, US

(21) Appl. No.: 12/639,498

(22) Filed: Dec. 16, 2009

Related U.S. Application Data
(63) Continuation of application No. PCT/EP2008/057769, filed on Jun. 19, 2008.
(60) Provisional application No. 60/945,172, filed on Jun. 20, 2007.

(51) Int. Cl. A61B 17/34

(52) U.S. Cl. 604/164.12

(57) ABSTRACT

An inserter for a medical device includes a housing, a carrier body that is movable relative to the housing. The carrier body further including a penetrating member defining a direction of insertion. The inserter including a second body that is movable relative to the housing and driving means that move respectively the carrier body and the second body relative to the housing. The driving means move the carrier body in the direction of insertion and the second body in a direction different from the insertion direction.

### CROSS-REFERENCE TO RELATED APPLICATIONS

This application is a continuation of PCT Application No. PCT/EP2008/057769 filed on Jun. 19, 2008, and which claims the benefit of U.S. Provisional Application No. 60/945,172, filed Jun. 20, 2007, all of which are incorporated herein by reference in their entirety.

### FIELD OF THE INVENTION

The invention relates to an inserter for a medical device e.g. an infusion set for intermittent or continuous administration of a therapeutical substance, such as e.g. insulin. The inserter comprises an insertion needle and a spring unit assuring automatic insertion and automatic retraction of the insertion needle.

### BACKGROUND OF THE INVENTION

It is known to construct inserters for infusion sets which hides and protects the insertion needle before insertion and which retracts the insertion needle after penetration of the patients skin and thereafter hides and protects the insertion needle.

Such a device is known from EP 1.762.259. The inserter according to this document comprises a needle hub comprising an insertion needle and two spring units assuring automatic insertion and automatic retraction of the insertion needle. Although the design of the device is compact and user friendly the mechanism is relatively complex as two spring units are used in order to make the device work correctly.

From US 2004/0158207 (Nunn et al.) is known a device for inserting a cannula into tissue, including a cannula (3), a protective element surrounding and accommodating the cannula and insertion needle before and after insertion, an operating element for moving the cannula out of the protective element, and a holder fixedly connected to the cannula. The spring (31) in the automatic embodiments (FIG. 13-18) of this document, pushes directly on the needle carrier (34) when the needle carrier is brought to a retracted position.

From WO 2006/061354 (Novo Nordisk NS) is known a medical device comprising a transcutaneous device unit and a process unit. The unit is adapted to be mounted to a skin surface of a subject and comprises a first housing, a transcutaneous device, and may comprise a flexible patch portion with an upper surface and a lower mounting surface adapted for application to the skin of a subject. The process unit comprises a second housing with a lower surface and a process assembly. The first and second housings are adapted to be secured to each other in such a way that the lower surface of the second housing is allowed to move freely relative to at least a portion of the underlying skin surface or patch. In this way a relatively flexible patch portion can adapt to the skin surface to which it is mounted both statically and dynamically without being restricted in its movements by the normally much stiffer process unit. FIGS. 25A-25D shows a schematic illustration of a multistep concept with a single loaded spring (1910) used to insert a cannula. When a first locking structure is released the cannula together with the insertion needles is inserted and a second locking structure (1912) is ready to be disengaged. When the second locking structure is disengaged the insertion needle is pulled away from the subject, and a third locking structure (1913) is ready to be disengaged. When the third locking structure is disengaged the cannula is pulled out. I.e. it is the intention that the inserter stays at the insertion position until the cannula has to be removed.

The present invention provides both protection of the insertion needle before insertion and after retraction and at the same time the inserter device is of a simple construction which only needs one spring unit.

### DESCRIPTION OF INVENTION

The object of the invention is to provide a simple, non-expensive inserter for an infusion device which inserter would be easy and safe for the user to handle during use and safe to dispose of after use.

An aspect of the invention concerns an inserter for inserting a medical device comprising a housing, a first body which is movable relative to the housing and comprising penetrating means pointing in the direction of insertion, a second body which is also movable relative to the housing and driving means which move respectively the first body and the second body relative to the housing wherein the driving means moves the first body in the direction of insertion and moves the second body in a direction different from the insertion direction.

In one embodiment the driving means first move the first body in the direction of insertion and then moves the second body in the direction different from the insertion direction. The driving means can comprise a single spring unit which could be a cluster of several springs or a single spring unit, e.g. a coiled spring. The spring unit of this one embodiment can work by first expanding in the insertion direction and then expanding in the direction different from the insertion direction. This direction different from the insertion direction can be in an angle of 180°±5° to the insertion direction.

In one embodiment the inserter device can be provided with first locking means which locking means can keep the first body in a chosen retracted position in relation to the housing while the driving means are biased i.e. the driving means posses stored or potential energy. These first locking means can comprise a protruding part on the first body which interacts with an opening in the housing.

In one embodiment the inserter is provided with second locking means which second locking means can keep the second body in a chosen forward position in relation to the housing while the driving means are biased i.e. the driving means posses potential energy, e.g. the second locking means comprises an inwardly protruding part of the housing which interacts with a distally turned surface of the second body.

According to one embodiment the inserter comprises means for locking the penetrating means to the second body while the second body is moving from a forward to a retracted position in relation to the housing.

The medical device used with the inserter device can be e.g. an infusion part for administrating medication or a sensor device for measuring values e.g. of the blood or a simple port/gateway for administering medication by a syringe or the like.

Another aspect of the invention also relates to a process for positioning a medical device on the skin of a patient which process comprises the following steps:
a) removing any packing and preparing the skin adhesion of the medical device;
b) placing the proximal end of the inserter against the skin of the patient or against a pre-positioned pad;.
c) unreleasing a first set of locking means (9) which will bring a first body (1) to reach a forward position and cause a proximal surface of the medical device to reach the surface it is to be placed on;
d) unreleasing a second set of locking means (11) which will result in that a second body (4) including parts locked to the second body (4) are distanced from the medical device which is left on the patients skin;
e) removing the inserter from the position while the medical device is left in the position.

### DESCRIPTION OF THE DRAWINGS

The invention is explained in greater detail below with reference to the accompanying drawings wherein preferred embodiments of the invention is shown.

FIG. 1 shows a cut-through view of an embodiment of a device according to the invention. The embodiment is in a first state where the penetrating member is retracted and the spring unit is tightened.

FIG. 2 shows the same embodiment as FIG. 1 in the same state but in a view which is perpendicular to the view of FIG. 1.

FIG. 3 shows the same embodiment as FIGS. 1 and 2 in a second state where the penetrating member is in a forward position and the spring unit in a semi-tightened state.

FIG. 4 shows the same embodiment as the previous figures in a third state where the penetrating member is in a retracted position; the medical device is left on the skin of the patient and the spring unit in a non-tightened state.

FIG. 5 shows an exploded view of the same embodiment as the previous figures.

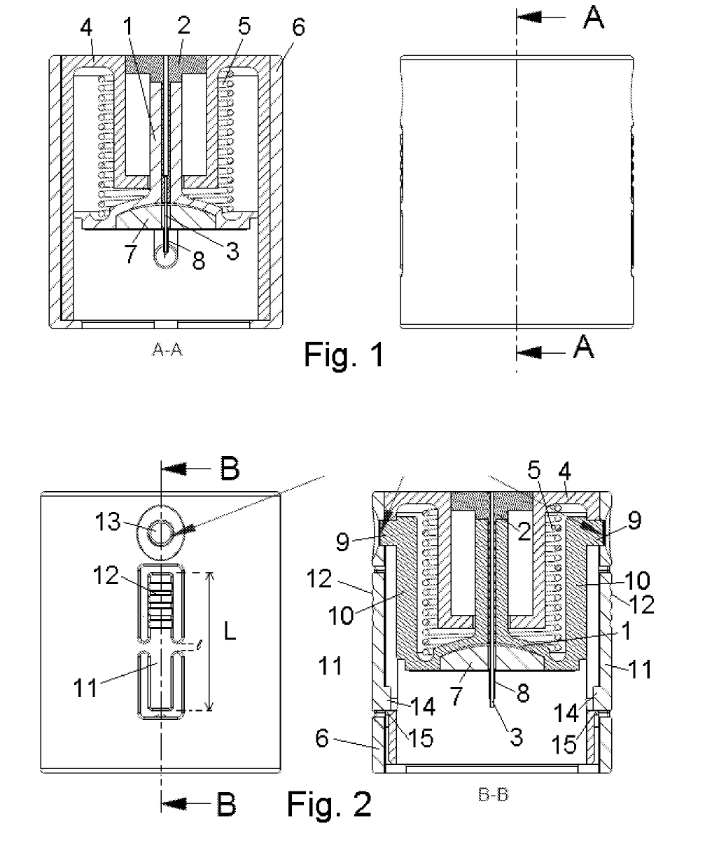

The device illustrated in FIG. 1 comprises a carrier body 1 for a medical device to be inserted, a needle hub 2 comprising a penetrating member 3, a movable part 4, a spring unit 5 and a housing 6. The spring unit 5 is tightened or biased in the space between the first body 1 and the second body 4, both of which are located in the housing 6.,

The medical device can be all sorts of devices which for some reason needs to be placed sub- or transcutaneously on a patient for a shorter or longer time. In the embodiment illustrated in FIG. 1-5 the medical device is a port-device. A port-device is a device which is placed on a user's skin for a period of up to three days and replaces numerous injections normally made be a syringe. The medical device could instead be an infusion part which device is also normally situated on the patient for several days and an infusion part has means for connecting it to a delivery device e.g. for delivering insulin. An infusion part can e.g. provide an optimized administration of a therapeutic substance as it allows very small doses to be transferred often. The medical device can also be a sensor device provided with a subcutaneously placed sensor which is in contact with the patient's blood and able to register desired elements in the blood e.g. the sugar level.

The port-device shown in the figures comprises a body 7 having a through going opening and a cannula 8 made of a soft and flexible material.

FIG. 2 shows the same embodiment in the same state as FIG. 1 but from an angle perpendicular to the view angle illustrated in FIG. 1. From this angle it is possible to see how the carrier body 1 is provided with two protruding locking parts 9 which are positioned at the end of each a flexible arm 10. The protruding locking parts 9 fit into corresponding openings in the stationary housing 6 and lock the carrier body 1 in a retracted position before use. The spring unit 5 is biased and pushes against the locked carrier body 1 (downwards on FIG. 2) and the movable part 4 (upwards on FIG. 2). When a user puts pressure on the opposite positioned pressure points 13 which are corresponding to the oppositely positioned protruding parts 9, the protruding parts 9 are disengaged from the openings in the housing 6 and the spring unit 5 will push the carrier body 1 downwards as the movable part 4 is still locked in relation to the housing 6 with secondary locking means.

The secondary locking means have the form of a longitudinal cut-out 11 having the length L. The longitudinal cut-out is fixed to the housing 6 by thin parts of material close to the middle section, the fastening parts have the length/width l and are symmetrically placed on each side of the cut-out 11. The length/width of the fastening parts are less than 10% of the total length of the cut-out 11, in the present embodiment the length/width of the fastening parts are approximately 6% of the length of the cut-out 11. The fastening parts are so relatively thin that it is possible to pivot the free ends of the cut-out 11 around the axis formed by the fastening parts. A first of the free ends 12 is provided with means which will make the user recognize these parts of the cut-out 11 as pressure points. In the embodiment in FIG. 2 these means comprises a series of flutes which can be felt by the user and recognized as a pressure point. When the user put pressure on the two opposite positioned first free ends 12, the first free ends 12 move towards the centre of the device while the second free ends moves outwards away from the centre of the device. The second free ends are provided with inwardly protruding parts 14 which are engaged with the edge 15 of an opening in the movable part 4.

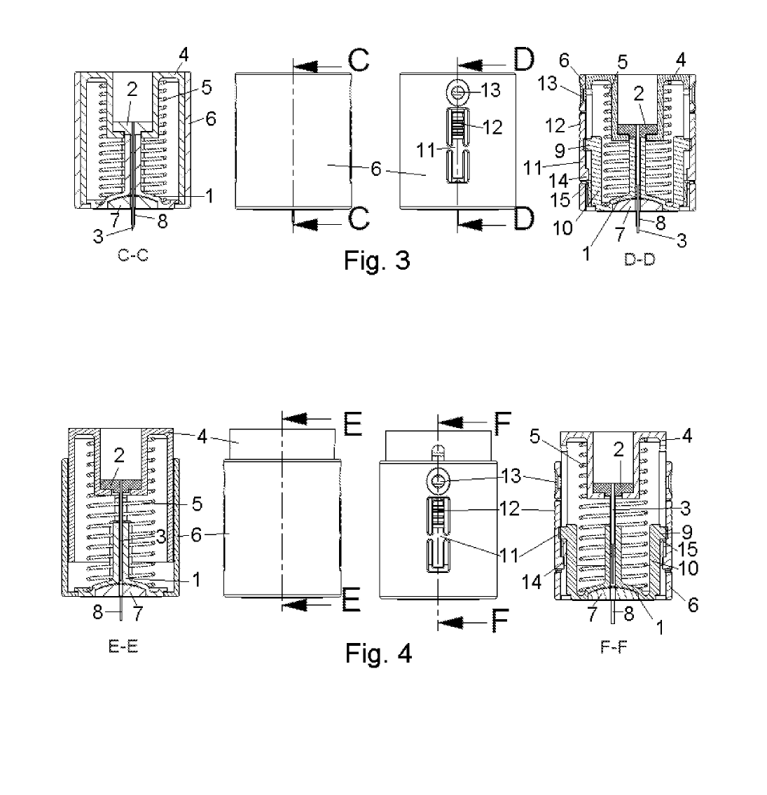

FIG. 3 shows two perpendicular views of the inserter device, the view C-C are similar to FIG. 1 and the view D-D is similar to FIG. 2. The spring unit 5 of the embodiment in FIG. 3 is in a semi-biased state, i.e. the spring unit 5 is released from the first lock made between the protruding parts 9 and the housing 6 and as a result of the releasing of the first locking means the cannula 8 of the medical device has been inserted subcutaneously in the patient. Also the carrier body 1 and the needle hub 2 has moved in relation to the movable part 4, the carrier body 1 has been distanced from the movable part 4 while the needle hub 2, which moves together with the carrier body 1, is moved from a surface position to an inner position in relation to the movable part 4 which makes it possible for the user to register that the penetrating member 3 is now in an inserted position.

FIG. 4 shows the same perpendicular views of the same embodiment of the inserter device as shown in FIG. 3, but the spring unit 5 of the embodiment in FIG. 4 is in an un-biased state, i.e. the spring unit 5 is also released from the second lock made between the inwardly protruding parts 14 of the housing 6 and the movable part 4 and as a result of the releasing of the second locking means the needle hub 2 together with the carrier body 1 is retracted from the patient. The user can observe this as the movable part 4 is raised in relation to the stationary housing 6.

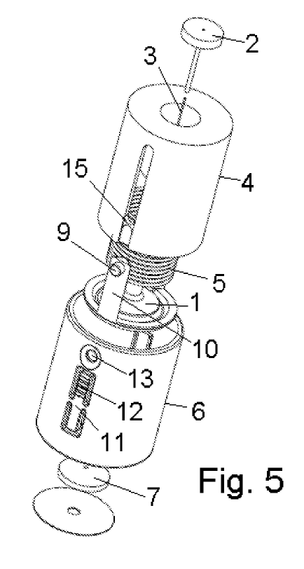

FIG. 5 shows the same embodiment as FIG. 1-4 but in an exploded view where the parts are partly separated in order to show the individual parts in a three-dimensional way and how the individual parts relate to each other. The parts have the same reference numbers as in FIG. 1-4.

The inserter device is normally delivered to the user joined with the medical device in a sterile packing which has to be removed before use. Either the medical device is provided with a mounting pad, that is a mounting pad is unreleasably fastened to the proximal side of the medical device, or a mounting pad is provided in a separate sterile packing and placed on the skin of the patient at a suitable insertion place before the medical device is inserted. If the mounting pad is unreleasably fastened to the medical device it will normally be necessary to remove a release layer from the adhesive surface of the mounting pad before using the inserter device.

After the sterile packing is removed and either the release layer is removed or the separate mounting pad is positioned, the inserter device is ready for insertion of the medical device.
The proximal end of the inserter device is then placed against the skin of the patient or against the pre-positioned mounting pad; the proximal end of the inserter device is the end towards which the sharp end of the penetrating member points.
Then the user puts pressure on the oppositely positioned pressure points 13 which will unlock the first locking means, i.e. the protruding locking parts 9. The unlocking of the first locking means result in the carrier body 1 including the medical device and the needle hub 2 being moved forward at the speed provided by the spring unit 5. The movement is stopped as the medical device touches the skin of the patient as further extension of the spring unit 5 is then prevented.
Then the user puts pressure on the oppositely positioned pressure points 12 which unlocks the second locking means, i.e. the pivotally fastened cut-out 11. The unlocking of the second locking means result in the movable part 4 including the needle hub 2 being pushed away from the carrier body 1 and the medical device which is left inserted into the patients skin.
The inserter device can now be removed from the insertion position while the medical device is left in the inserted position.

1. An inserter device for a medical device comprising:
a housing;
a carrier body for the medical device to be inserted, the carrier body is movable relative to the housing and comprises a penetrating member defining a direction of insertion, and a first locking member configured to releasably hold the carrier body relative to the housing;
a second body movable relative to the housing; and
driving means which move respectively the carrier body body and the second body relative to the housing; the driving means comprising a single spring unit placed between the carrier body and the second body, the driving means configured to first move the carrier body in the direction of insertion and then to move the second body in a direction which is in an angle of about 180°±5° to the insertion direction to distance the second body from the carrier body;
wherein the first locking member releasably holds the carrier body in a retracted position relative to the housing while the driving means are in a biased state.

2. An inserter according to claim 1, wherein the spring unit is in an un-biased state after moving the second body.

3. An inserter according to claim 1 wherein the spring unit is a coiled spring.

4. An inserter according to claim 1, wherein the carrier body is provided with two oppositely positioned protruding locking parts that are positioned at an end of each a pair of flexible arms, the protruding locking parts fit into corresponding openings in the housing and releasably lock the carrier body in a retracted position before use.

5. An inserter according to claim 4, wherein the housing further comprises two oppositely positioned pressure points corresponding to the oppositely positioned protruding parts, the protruding parts disengage from the openings in the housing in response to pressure on the pressure points and the spring unit biases the carrier body in the direction of insertion.

6. An inserter according to claim 1, wherein the inserter comprises a second locking member configured to hold the second body in a forward position relative to the housing while the driving means are in the biased state.

7. An inserter according to claim 6, wherein the second locking member comprises an inwardly protruding part of the housing configured to interact with a distally turned surface of the second body.

8. An inserter according to claim 6, wherein the second locking member comprises a longitudinal cut-out having a length L, a portion of the longitudinal cut-out is fixed to the housing by fastening parts so that free ends of the cut-out pivot around an axis formed by the fastening parts, the fastening parts have a length l and are symmetrically placed on each side of the cut-out.

9. An inserter according to claim 8, wherein the length l of the fastening parts is less than about 10% of the total length of the cut-out.

10. An inserter according to claim 8, wherein the length l of the fastening parts is less than about 6% of the total length of the cut-out.

11. An inserter according to claim 9, wherein a first of the free ends of the cut out comprises a tactile indicator for a pressure point.

12. An inserter according to claim 11, wherein the tactile indicator comprises at least one flute.

13. An inserter according to claim 1, wherein the inserter comprises means for locking the penetrating member to the second body while the second body is moving from a forward to a retracted position relative to the housing.

14. An inserter according to claim 1, wherein the medical device is an infusion part for administrating medication or a sensor device for measuring values or a port for administering medication.

15. An inserter according to claim 1, further comprising a first pair pressure points corresponding to oppositely positioned protruding parts and configured to disengage the carrier body from the housing and a second pair of pressure points configured to to disengage the second body from the housing.

16. The inserter according to claim 1 wherein the spring unit is positioned concentrically between the carrier body and the second body.

17. A method for positioning a medical device on the skin of a patient, the method comprising:
placing a proximal end of an inserter against the skin of the patient;
releasing a first set of locking members of the inserter to bring a carrier body of the inserter to a forward position and cause a proximal surface of the medical device to reach the surface of the skin;
releasing a second set of locking members of the inserter to distance a second body including parts locked to the second body from the medical device which is left on the patient's skin;
removing the inserter from the surface of the skin while the medical device is left on the patient's skin.

18. The method of claim 17 further comprising positioning a pad on the patient's skin before placing the proximal end of the inserter against the skin.
